(19) United States
(12) Patent Application Publication
(10) Pub. No.: US 2024/0299992 A1
(43) Pub. Date: Sep. 12, 2024

(54) METHOD FOR CLEANSING HUMAN KERATIN MATERIALS, DEVICE AND KIT FOR PERFORMING SAID METHOD

(71) Applicant: L'OREAL, Paris (FR)

(72) Inventors: Dominique BORDEAUX, Chevilly-LaRue (FR); Laure DESFORGES, CHEVILLY-LARUE (FR)

(21) Appl. No.: 18/254,682

(22) PCT Filed: Nov. 24, 2021
(86) PCT No.: PCT/EP2021/082878

§ 371 Date: May 26, 2023

(30) Foreign Application Priority Data
Nov. 26, 2020 (FR) 2012157
Nov. 26, 2020 (FR) 2012163

(51) Int. Cl. B08B 7/04; A61K 8/44; A61K 8/46; A61K 8/73; A61Q 19/10; B08B 3/08; B08B 3/12; B08B 7/02

(52) U.S. Cl. CPC B08B 7/04; A61K 8/44; A61K 8/463; A61K 8/731; A61Q 19/10; B08B 3/08; B08B 3/12; B08B 7/026; A61K 2800/28

(57) ABSTRACT

Method for cleansing external human keratin materials in contact with a cosmetic composition within which bubbles of a gas are present and/or are generated, the method including the step consisting in subjecting bubbles of the gas, present in the cosmetic composition in the region of the surface of said materials to be cleansed, to acoustic waves in order to bring about their collapse and to generate a mechanical shock on the surface to be cleansed in order to remove the soiling therefrom.

### TECHNICAL FIELD

The present invention relates to the cleansing of human keratin materials.

### PRIOR ART

Patent application JP 2007-311756 describes an ultrasonic cleaning device for cleaning a silicon wafer, a mask substrate, etc. The device comprises an ultrasonic transducer mounted in a nozzle through which passes a cleaning liquid, and a means for introducing a gas so as to generate bubbles in the cleaning liquid.

Patent application US 2012/0227761 describes a device for cleaning a surface, including firstly a chamber fed with a liquid and communicating with an outlet pipe emerging on the surface to be cleaned, an ultrasonic transducer transmitting acoustic energy to the liquid contained in the chamber and the outlet pipe, and secondly a bubble generator for generating bubbles of a gas in the outlet pipe. The bubble generator may be an electrochemical generator, the liquid containing, for example, a salt such as potassium chloride to make the liquid electrically conductive. A surfactant may be added to prevent the coalescence of the bubbles during their path in the outlet pipe towards the surface to be treated, and to ensure that they have the required size when they reach the surface to be treated.

Application of the device for cleansing the skin, notably underneath a surgeon's nails, and also the hands, is envisaged.

Said patent application mentions the possible addition of surfactant to influence the size of the bubbles produced.

No cosmetic application for treating facial skin or the hair is disclosed.

Patent applications US 2017/0080257, KR 2020/0102956, US 2009/318853, JP 2016/214424 and WO 2020/029429 disclose cosmetic methods for treating the skin, notably for cleansing it, using an ultrasonic device which generates microbubbles in a cosmetic composition applied to the skin to be cleansed.

Similar devices are disclosed in patent applications US 2011/213281 and US 2010/010420.

These devices use a large amount of water and of fresh compositions on each use, the whole being discarded.

The ecodesign of products by promoting the sustainable use of resources has become a key factor essential for minimizing the impact of products on the environment. Producers are bearing the burden of responsibility and are being encouraged by the changes in consumption to ecodesign their formulations and packagings, while at the same time taking care to optimize the industrial methods and to manage the production waste. A virtuous circle is thus established. Similarly, accompanying consumers towards greater sobriety pertains to this global movement towards individual responsibility. A product with a reduced environmental footprint can thus echo consumers' increasingly genuine expectations.

### DISCLOSURE OF THE INVENTION

The invention is directed towards proposing a method for cleansing human keratin materials, in particular facial skin or the hair, enabling said materials to be efficiently cleansed, and falling within a responsible sustainable development approach, by means of a reduced carbon footprint.

### SUMMARY OF THE INVENTION

One subject of the invention is thus a method for cleansing external human keratin materials in contact with a cosmetic composition within which bubbles of a gas are present and/or are generated, the method including the step consisting in subjecting bubbles of the gas, present in the cosmetic composition in the region of the surface of said materials to be cleansed, to acoustic waves in order to bring about their collapse and to generate a mechanical shock on the surface to be cleansed in order to remove the soiling therefrom.

This method is preferably cosmetic and, notably, non-therapeutic.

The method according to the invention is preferably intended for topical application.

A subject of the invention is also a device for cleansing external human keratin materials, including an applicator arranged to dispense a cosmetic composition on an area to be treated, via at least one outlet, a chamber in which the composition circulates and at least one ultrasonic transducer to emit acoustic waves in the chamber, the transducer being powered by a current generator, and a bubble generator to generate within the composition gas bubbles which will be subjected to the acoustic waves emitted by the transducer; the device also includes a system for the at least partial recovery and recycling of the cosmetic composition sent in contact with the keratin materials.

The cosmetic composition may be reused after it has passed through the recovery and recycling system.

Preferably, the device is a cosmetic device, notably for non-therapeutic purposes, better still for topical application of a cosmetic composition.

The term “cosmetic composition” denotes a composition containing at least one cosmetic active agent, as defined in the Cosmetics Directive 76/768/EEC.

According to the invention, mineral water or running water does not constitute a cosmetic composition.

The term “external human keratin materials” denotes the skin and its integuments, notably the hair and the nails, and excludes mucous membranes, for example the gums. The area of skin that is treated according to the invention may be the skin of the face, neckline, back, arms, legs, hands and/or feet, and the scalp. The method according to the invention is most particularly suitable for removing makeup from and/or cleansing facial skin, notably of the forehead, cheeks, chin, nose and scalp.

Among the undesirable soiling that may be present on the keratin materials (at the surface and/or anchored more deeply in the skin pores), exogenous impurities such as makeup, environmental pollution, dust, microorganisms, etc., and endogenous impurities or defects such as excess sebum, sweat, dead cells, dead skin, dandruff, blackheads, light scars and/or acne scars, pigmented marks, etc., may be distinguished.

The method or the device according to the invention makes it possible to efficiently remove or treat both the exogenous impurities and the endogenous impurities or defects. Under the effect of the acoustic waves, the bubbles generate shock waves which can exert a mechanical effect on the keratin materials. They can also contribute towards the release of chemical species which contribute towards the cleansing of the keratin materials, such as free radicals.

The term “in the region of the surface of said materials to be cleansed” should be understood as meaning close enough for the effect of the collapse of the bubbles under the action of the acoustic waves to have a beneficial effect on the cleansing action. For example, bubbles are present less than 5 mm from the surface to be cleansed, better still less than 2 mm and even better still less than 1 mm.

In accordance with the invention, the composition advantageously has a total content of cleansing active agent(s) of at least 0.02% by mass relative to the total weight of the composition. With a total content of cleansing active agent(s) of at least 0.02% by mass, better still at least 0.05% and even better still at least 0.1%, relative to the total weight of the composition; thus, the composition can also have a cleansing action in the absence of collapse of the bubbles by the acoustic waves. This content may be less than or equal to 20% by weight.

The cleansing active agents may contribute towards the formation of the bubbles and/or towards their stabilization, these agents being chosen, for example, from the compounds detailed later, notably from foaming surfactants such as polyoxyalkylenated alkyl(amido) ether carboxylic acid anionic surfactants, anionic surfactants different from the abovementioned polyoxyalkylenated alkyl(amido) ether carboxylic acids, nonionic surfactants, amphoteric and zwitterionic surfactants, and mixtures thereof, and/or are chosen from the compounds conventionally present in makeup-removing compositions such as alkylpolysaccharides, fatty alcohol polyethylene glycols, oils, and mixtures thereof.

### Acoustic Waves

The acoustic waves are generated by one or more transducers, notably ultrasonic transducers. The acoustic waves are preferably ultrasonic waves.

The frequency of the acoustic waves ranges, for example, from 3 kHz to 5 MHZ, better still from 3 kHz to 1 MHz, even better still from 10 kHz to 1 MHz, notably from 10 kHz to 500 KHz.

The power of the acoustic waves ranges, for example, from 30 mW to 100 W/cm² (with, in the case of high power, a duration that is sufficiently short so as not to cause damage to the keratin materials, for example a duration of less than or equal to 1 s), better still from 0.1 to 10 W/cm², or even 1 to 7 W/cm².

The acoustic waves may be generated by a single transducer or, as a variant, by at least two transducers. Each transducer may include a sonotrode, typically made of metal, which can define a surface via which the acoustic waves are emitted. Such a sonotrode may be made of metal, for example. The transducer may include at least one piezoelectric material.

The acoustic waves may be generated permanently as soon as the treatment device is switched on or, as a variant, may be generated only when certain operating conditions are met, for instance the presence of the composition in contact with the transducer(s) and/or the presence of the device in contact with the area to be treated and/or if the device detects that the area bears soiling.

The acoustic waves may be generated by a sinusoidal signal or by a signal of more complex shape, for example with frequency modulation or with amplitude modulation. The acoustic waves are preferably emitted at a single frequency, which may enable them to be focused more precisely on a given area, but, as a variant, may be emitted at several different frequencies. The acoustic waves may be emitted continuously or in the form of pulses.

The method may thus include a step of detecting the presence of the cosmetic composition on contact with the transducer(s), and condition the functioning of the transducer(s) to this detection.

Similarly, the device may include a system for detecting the presence of the cosmetic composition on contact with the transducer(s), and condition the functioning of the transducer(s) to this detection.

The method may also include a step of detecting the presence of the device on contact with the area to be treated, and condition the functioning of the transducer(s) to this detection.

Similarly, the device may include a system for detecting the presence of the device on contact with the area to be treated, and condition the functioning of the transducer(s) to this detection.

The emission of acoustic waves in the absence of composition or when the device is not in the situation of treating keratin materials is thus avoided.

Preferably, the acoustic waves are emitted without the transducer or the associated sonotrode coming into contact with the area to be treated. The acoustic energy emitted in the keratin materials is thus limited.

A composition can be circulated continuously in contact with the transducer(s), with continuous functioning of the transducer(s), or, as a variant, pulsed functioning of the transducer(s). The composition can also be fed in a pulsed manner. The feeding of an area subjected to the acoustic waves is advantageously discontinuous, for example with a period of feeding the area while the acoustic waves are not generated, and then a period in which the composition present in the area is subjected to the acoustic waves without being refreshed during this exposure. This makes it possible to prevent the bubbles present in the composition before entering said area from being repelled by the acoustic waves present therein.

It is thus possible to servocontrol the feeding with composition to the functioning of the transducer(s) for the production of the acoustic waves or vice versa, and to have pulsed functioning of the transducer(s) and/or of the feeding with composition. In other words, supplying of composition that is offset in time relative to the period of emission of the acoustic waves into the area into which the composition is supplied may be performed.

The propagation of the acoustic waves may be facilitated by the presence of liquid in the composition. The gas/liquid mass ratio in the composition can thus benefit from not being too high, and the composition may advantageously contain a thickener, as detailed hereinbelow, or any other compound for increasing the temporal stability of the bubbles.

The transducer(s) used to generate the acoustic waves may have an area in contact with the composition which ranges, for example, from the area of a circle 5 mm in diameter to a circle 100 mm in diameter, better still from the area of a circle 5 mm in diameter to that of a circle of 50 mm, or better still from the area of a circle 5 mm in diameter to that of a circle of 40 mm.

The sonotrode may be given any form suited to that of the surface to be treated, where appropriate.

The distance between the transducer(s) for generating the acoustic waves and the area to be treated may, if necessary, correspond substantially to the diameter of a bubble and preferably ranges from 100 microns to 40 mm, better still from 100 microns to 10 mm.

### Gas Bubbles

The gas bubbles may be bubbles of air, CO₂, oxygen, hydrogen or nitrogen, among other possibilities, and also a mixture of these gases.

All the bubbles may be bubbles of the same gas or, as a variant, the composition may include bubbles of a first gas and bubbles of a second gas which is different from the first.

The gas may originate from decomposition of the composition or may be extracted therefrom, or, as a variant, may be introduced into the composition.

The diameter of the bubbles may range from 100 nm to 700 μm, better still from 500 nm to 50 μm. The size denotes herein the number-average size D50 of half of the population.

The dimensionless factor γ equal to the ratio d/Rₘₐₓ, where d is the distance from the geometrical centre of the bubble to the surface to be cleansed when the expansion of the bubble is maximal, and Rₘₐₓ is the maximum expansion diameter of the bubble, is preferably less than 3.5, better still less than 1.1, so as to have maximum efficiency, as detailed in the publication Mechanisms of single bubble cleaning, F. Reuter, Ultrasonics Sonochemistry 29(2016) 550-562.

The density of the medium formed by the cosmetic composition with the gas bubbles, exposed to the acoustic waves, may be between 0.1 and 1 g/cm³, better still between 0.5 and 1 g/cm³ (at 20° C. and at atmospheric pressure).

A small bubble size may facilitate their penetration into the skin follicles, such as the hair follicles, and thus exert efficient cleansing action therein. It may thus be advantageous for these bubbles to be less than or equal to 300 microns and better still 200 microns in size, for example 100 microns or less in size. The bubbles can then enter the follicles before being activated by the acoustic waves.

### Bubble Generation

The bubbles may be generated by any suitable means, for example a mechanical, physical, chemical or electrochemical means. The bubbles may notably be generated by a negative pressure in the liquid, which makes it possible to lower the vapour pressure and to generate the formation of gas in the form of bubbles.

The bubble generation may be prior to, simultaneous with or cyclic relative to the emission of the acoustic waves.

As examples of techniques that may be performed for generating the bubbles in the invention, there are, inter alia, the techniques:
of decompression of liquids using nozzles, for example, the decompression possibly being preceded by pressurization,
of creation of a turbulent flow, notably using rotating blades, a turbomixer, an ejector, a Venturi, a turbulent gas or liquid flow, a rotating porous body, notably a rotating disc, a generator as described in the publication “Performance of a new micro-bubble generator with a spherical body in a flowing water tube”, M. Sadatomi, Experimental Thermal and Fluid Science 29 (2005) 615-623, a Venturi tube and a vortex, a Venturi as used by the company MEC Co. (lona Shower), or shear forces,
of creation of a laminar flow through small apertures, with focusing or co-focusing of the flow, of a porous membrane, using a porous-membrane fluidic oscillator as described, for example, in the article “Fluidic oscillator-mediated microbubble generation to provide cost-effective mass transfer and mixing efficiency to the wastewater treatment plants”, Environ. Res. 2015 February; 137: 32-9, of an emulsification microchannel as described in the article “Monodispersed microbubble formation using microchannel technique”; AIChE Journal, 50 (2004), pages 3227-3233, these bubble creation techniques by laminar flow through small apertures being preferred to the preceding techniques on account of the stability of the bubbles produced,
of supplying energy:
   light energy, for example by irradiation of microfluidic channels covered with nanoporous gold, as described in Progress in Biomedical Optics and Imaging Proceedings of SPIE, volume 9705, 2016 Article number 97050D, “Photothermal generation of microbubbles on plasmonic nanostructures inside microfluidic channels” (Conference paper), by laser irradiation, for example as described in “An experimental study on microbubble generation by laser induced breakdown in water”, The review of Laser Engineering (Suppl.) (2008), pages 1273-1275 (2), by laser irradiation on carbon nanotubes, as described in the article “Producing single microbubbles with control size using microfiber”, Advance in Bioscience and Biotechnology, 2 (2011), pages 385-390, by a tapered optical fibre, by a plasmonic absorber in the near infrared, as described in the publication “Size-controllable micro-bubble generation using a nanoimprinted plasmonic nanopillar array absorber in the near-infrared region”, Applied Physics Letters, 108, 2016,
acoustic energy, for example by ultrasound as described in the article Ultrasonics Sonochemistry, volume 29, 1 Mar. 2016, pages 604-611, “Influence of sonication conditions on the efficiency of ultrasonic cleaning with flowing micrometer-sized air bubbles”, sonic activation of microbubbles, ultrasound in the presence of nucleation sites, as described in the patent application WO 2016/055883,
electrical energy, for example via an electrodynamic coaxial atomization method as described in the publication “Preparation of microbubble suspensions by co-axial electrohydrodynamic atomization”, Medical Engineering and Physics, 29(2007), pages 749-754, by electrolysis, electroflotation, by an electrolytic microgenerator as described in the publication “Micro-fabricated electrolytic micro-bubblers”, International Journal of Multiphase Flow, 31 (2005), pages 706-722, by electrostatic spraying as described in the publication “Microbubble generation for environmental and industrial separations”, Separation and Purification Technology, 11 (1997), pages 221-232, by electrical heating of carbon nanotubes as described in the publication “Microbubble generation with micro-watt power using carbon nanotubes heating elements”, Proceedings of the 7th IEEE International Conference on Nanotechnology, Aug. 2-5, 2007, Hong Kong (2007) or by microplasma, as described in the article Journal of Physics D; Applied Physics, volume 47, issue 35, 3 Sep. 2014, article number 355203; “Microbubble generation by microplasma in water”.

The bubbles may be continuously generated, as soon as the device is switched on. As a variant, the bubbles are generated intermittently, for example only when the composition is dispensed, or periodically at a predefined frequency so as to give them time to disperse. A certain amount of bubbles may be present in the composition before the device is switched on.

The intensity of the bubble production may be constant or variable and, where appropriate, adjustable by the user or automatically by the device as a function of the desired result or of at least one operating parameter.

The bubbles may be generated by any of the techniques mentioned above, in particular by injecting a pressurized gas into the composition, for example using a pump or a compressed gas tank, by electrolysis of the cosmetic composition, by blending of the composition, suction of a gas into the composition, or vaporization of a liquefied gas mixed with the composition or dissolved in the composition. The bubbles may originate from the reaction of two liquids or of one liquid and of at least one solid in powder, granule or tablet form, for example, or any other form.

The flow of liquid containing the bubbles may range from 0.01 mL/s to 10 mL/s.

### Abrasion

The method may include abrasion of the keratin materials using abrasive particles and/or a part of the device in contact with the keratin materials.

The abrasion may be performed prior to the exposure of the cosmetic composition and the bubbles to the acoustic waves, or, as a variant, concomitantly. When the abrasion takes place beforehand, it may be performed via any means, notably by mechanical or chemical action.

In this case, the abrasion of the keratin materials may be caused not only by the shock wave which is produced following the exposure of the bubbles to the acoustic waves, but also at least partly by the action of the abrasive particles impacting the surface to be treated, for example the particles present on the surface of the applicator coming into contact with the external keratin materials.

The abrasive particles present in the cosmetic composition may be insoluble in the medium of the composition or, as a variant, soluble therein, and can then preferably generate a gas during their dissolution, which gas will then serve to generate all or some of the bubbles subjected to the acoustic waves.

The abrasive particles may be chosen from the abrasive powders of materials having a hardness on the Mohs scale of greater than or equal to 3, for example powders of alumina, silica, aluminosilicates or carbonates, or of a material coated with a silica, an alumina or an aluminosilicate.

It may also be a powder of fruit kernels, notably of apricot, of wood cellulose, for example of ground bamboo stalk, of coconut husk, or a synthetic material such as polyamide, or mixed particles combining organic and inorganic compounds, and particles coated with the above compounds.

The abrasive particles may have a size of between 0.1 and 500 microns, notably between 0.1 and 50 microns, for hair treatment, and between 10 and 300 microns for treatment of the scalp or of facial skin.

The solid particles used to exert an abrasive action may have a flattened, spherical, elongated, polyhedral or irregular shape.

### Recycling

Preferably, the cosmetic composition is sent into contact with the keratin materials so as to be at least partially recovered in order to be recycled.

The composition that is recovered may be filtered to be freed of the solid debris or of its particulate phase, before being sent again to the surface to be cleansed.

Preferably, the composition is recovered by suction or by absorption, for example using a porous support.

Advantageously, the treatment and purification unit comprises a treatment unit inlet for receiving a spent composition comprising the composition which has been in contact with the keratin surface, and a separator comprising a slurry collection bowl and a liquid collection chamber.

More advantageously, a spray nozzle is mounted inside the bowl.

Even more advantageously, the spray nozzle includes a spray nozzle inlet and a spray nozzle outlet, the treatment unit inlet communicating with the spray nozzle inlet and the spray nozzle outlet emerging in the slurry collection bowl.

According to an advantageous embodiment, a bowl lid separates the slurry collection bowl from the liquid collection chamber, a channel passing through the bowl lid to discharge the composition freed of the slurry in the liquid collection chamber. The two chambers may be filled and emptied via an aperture (lid). They may be totally hermetic and disposable and/or collectable to be cleaned via a dedicated network and subsequently refilled.

Advantageously, an outlet from the centrifugal separator communicates with the liquid collection chamber and the centrifugal separator is capable of separating the spent liquid from the slurry it contains by centrifugation of the slurry-charged water which has a higher density than that of the water on the walls of the slurry collection bowl.

The liquid collection chamber also advantageously comprises a deflector which diverts the liquid gradually as it enters the chamber, said deflector preventing turbulence from accumulating in the chamber. The chamber may include a nonwoven filter made of natural or artificial fibres, filter particles such as sand, silica, or any other means for retaining the matter in suspension.

According to a particular embodiment of the invention, the treatment and purification unit includes a disinfection unit which kills bacteria in the recycled composition, said disinfection unit comprising a UV lamp and/or an ozone or oxidant generator and/or a chemical product or bactericide dispenser and/or a heating device.

The treatment and purification unit advantageously comprises an optical contaminant detection unit intended for measuring an absorption at at least one wavelength and comprising at least one source for generating one or more optical signals at one or more wavelengths, at least one detector for detecting said optical signal(s) at one or more wavelengths and for emitting electrical signals indicating the presence of said contaminants.

Preferably, the treatment and purification unit comprises a filter having a pore size of less than or equal to 50 microns. The filter may be a nonwoven fabric, with or without folds.

The filter may be formed by a filter system equipped with a nanofilter and at least one filter quality sensor, and at least one prefilter, the prefilter being positioned before the nanofilter in method terms, and the filter quality sensor being provided to indicate that the filter system is functioning satisfactorily.

More preferentially, the optical detection takes place upstream of said filter so that the contaminants which cannot be filtered cannot enter said recycling pipe.

More advantageously, the unit includes a return pump to suck up the spent composition passing through the centrifugal separator, comprising a pump inlet and a pump outlet, the pump inlet being connected to the centrifugal separator outlet.

Even more advantageously, the unit includes a filtration device comprising a filtration device inlet, the filtration device inlet being connected to the pump outlet, a treatment unit outlet being connected to the filtration device outlet.

Preferentially, the unit includes a feed pump feeding the unit inlet with water at a feed rate, in which the return pump sucks up the water at a return rate, in which the feed rate is less than the return rate, so that the return pump sucks up both water and air through the centrifugal separator.

Advantageously, the recycling unit is configured so as not to release into the external environment the solvent in vapour form, or so as to release only a small amount thereof, for example by means of recycling of the solvent, the recycling taking place, for example, in vapour or liquid form, after condensation of the solvent.

### Chamber

The capacity of the chamber is, for example, between 5 and 100 ml, better still between 7.5 and 50 ml.

Preferably, the chamber and the system for feeding the applicator member with the composition belong to one and the same refill-forming assembly, which is able to be handled in one piece in order to be fitted on the device and removed therefrom; refilling with product is thereby made easier.

The refill-forming assembly may be designed to be fixed, preferably by click-fastening, to the handpiece, for example to a casing in which the electrical generator is contained.

The refill may include one or more cartridges, in which different compositions may be stored. These compositions may be mixed at the time of use and may, for some of them, be solid and/or in the form of powders that are rapidly soluble.

Advantageously, the cosmetic device according to the invention includes a cosmetic composition refill, which may or may not be removable.

### Applicator

The applicator may include a porous material and/or a material that is able to release or diffuse the cosmetic product, notably an open-cell foam, preferably borne by a removable support, notably in the form of a frame.

Thus, the applicator may include dispensing orifices such as slots, which are closed at rest and open under the pressure of the upstream composition, and have an elasticity that allows an increase in volume during filling and dispensing of composition that continues after the end of the filling action.

The applicator may include an applicator member mounted, where appropriate, in a removable manner on the device; as a variant, an applicator member may be fixed to the applicator. Where appropriate, the device may include means that make it easier to eject the applicator member in order to replace it, such as an ejector pushbutton. The applicator may include a chamber for storing a sufficient amount of composition to allow the cleansing and the contact of the bubbles with the surface to be cleansed. The applicator may be designed from flexible and/or rigid materials. The applicator must make it possible to prevent leaks of composition intended for cleansing. The applicator may be removable and can be closed to enable the composition to circulate in a loop.

### Application of the Cosmetic Composition

In general, the composition may in itself, by virtue of its formulation, already contribute towards the removal of the impurities that it is desired to remove by the action of the bubbles subjected to the acoustic waves. The action of the bubbles subjected to the acoustic waves can accelerate or improve this method. Thus, the association of the wave generated by the collapse of the bubbles in combination with the action of the combination may, by synergy, have an effect greater than that of the waves alone or than that of the composition alone.

The cosmetic composition may be applied in a first stage, with bubbles already present therein, and then exposed in a second stage, after it has been applied to the keratin materials, to the acoustic waves in order to bring about shock waves following the collapse of the bubbles.

For example, the user first applies a cosmetic composition in foam form to the area to be cleansed, for example by spraying it onto said area, and then brings a treatment device into contact with the composition, to subject said composition to the acoustic waves.

The cosmetic composition may also be applied otherwise, and notably continuously, i.e. a circulation of the composition is established on contact with the keratin materials to be treated, this circulation being, for example, in a closed or open circuit.

When the circulation takes place in a closed circuit, the composition is at least partially recycled. An additional amount of composition may be introduced continuously or intermittently into the circuit to compensate for the losses.

The circulation can be stopped at any moment to promote prolonged static contact of the composition on the surface to be cleansed. The circulation may take place without the presence of bubbles, allowing a first contact with the keratin material to prepare it for the cleansing, for example.

In an open circuit, the composition is not recycled for performing the method, and is, for example, removed to a collection tank or directly with the waste water.

The circulation of the composition takes place, for example, with a flow rate of between 0.01 mL per second to 5 mL per second.

The bubbles may be formed while the cosmetic composition is already in contact with the keratin materials, by means of a bubble generator of the device, for example functioning by electrolysis.

### Handpiece

The method may be performed with a handpiece to be placed in contact with the keratin materials to be treated.

This handpiece may carry at least one ultrasonic transducer for generating the acoustic waves and may be arranged to keep an acoustic wave emission surface at a distance from the surface to be treated.

The handpiece may include at least one flexible lip acting as a spacer to maintain this separation. This flexible lip may also participate in confining the cosmetic composition in a space located between the surface to be treated and the transducer. This flexible lip may contribute towards dispensing, spreading, collecting and/or recycling the composition.

The cosmetic composition may be made to circulate in a space, which may be modulable, located between a sonotrode of the handpiece and the area to be treated.

This circulation may take place continuously, as mentioned above, or intermittently. Said space may also be filled with the composition while the handpiece is in place, followed by generating the acoustic waves in the composition filling this space.

### Selection of the Treated Areas

The method may be performed on all or part of the skin of the face, scalp or body, to cleanse same.

The treated area may notably be an area of keratin materials covered with a makeup product such as a foundation, a lipstick, a blusher, a mascara, an eyeliner, a powder, an emulsion, an oil or an antisun product, inter alia, and the treatment may be directed towards removing this product.

The method may be performed by moving a handpiece along the area to be treated, so as to treat the entire surface covered with a makeup product or any substance that it is desired to remove.

The method may also be performed to treat an area of skin not coated with a makeup product, so as to cleanse it deep down, and to remove or treat exogenous or endogenous impurities or defects, for example dead skin cells, traces of sebum or sweat, dandruff, bacteria, traces of pollution, blackheads, pigmented marks, light scars or acne scars. The method may be performed to treat the scalp. The method may also be performed on the top of the nails to remove a nail varnish.

The method may also be performed to cleanse the hair, notably in order to at least partially remove a colouring performed previously.

### Related Methods

The method according to the invention may be preceded or followed by a cosmetic method such as making up or massaging.

For example, the skin, the hair or the eyelashes are made up, and the makeup is then removed after a certain time (for example less than 24 hours) by means of the cleansing method according to the invention.

In another example, the skin is cleansed by performing the method according to the invention, and a care treatment is then performed on the cleansed area (for example just after or less than two hours after), for example a massage and/or the application of a care composition.

### Treatment kit

A subject of the invention is also a treatment kit for performing the method according to the invention, as defined above.

This kit comprises:
the cosmetic composition in which the bubbles are generated,
a device for exposing the bubbles to acoustic waves in the region of the surface to be treated.
The composition may be packaged with the device in the same packaging. Where appropriate, the composition is contained in a container arranged to be mounted on the device, this container constituting, for example, a cartridge arranged to be totally or partly fixed into a corresponding housing of the device, or to be connected to the device via a suitable connection such as a hose.

### Cosmetic Composition

The composition may generally include any compound conventionally included in the formulation of compositions for cleansing and/or caring for human keratin materials, which is compatible with the generation of bubbles that are sufficiently stable before the application of the acoustic waves.

### Stabilization of the Bubbles

The cosmetic composition preferably contains at least one compound with a stabilizing action, such as surfactants, particulate compounds, salts, polymers or any type of compound which makes it possible to increase the lifetime of the bubble in its medium, contributing towards stabilizing the bubbles, i.e. making it possible to prevent them from coalescing prematurely, before being subjected to the acoustic waves, while at the same time being close to or in contact with the surface to be treated. The time between the moment when the bubbles are generated and the moment when they are subjected to the acoustic waves ranges, for example, from a few milliseconds to a few days. It is known that several factors promote bubble formation and stability:
The amount of surfactant: the surfactants will preferably be available in the composition in the form of micelles, and thus at a concentration above the critical micelle concentration (CMC: concentration at and above which the surfactants associate to form micelles) including once the bubbles have been formed. To obtain this, the concentration of the surfactant in the product is preferably from 5 to 10 times the CMC.
The viscosity of the medium: a more viscous medium makes it possible to obtain more stable bubbles. For example, the use of polymers (e.g.: proteins) and/or of thickeners makes it possible to obtain this.
The hardness of the water: the foaming power of anionic surfactants is generally weakened in hard waters on account of the Ca2+ and Mg2+ ions which interact with the surfactants.
The nature of the gas used: the bubbles will be more stable if the gas used is sparingly water-soluble.

This compound with a stabilizing action may also exert an action on the surface to be treated, notably a mechanical, chemical and/or biological action.

The bubbles may be generated and/or stabilized by any suitable means, for example by using foaming surfactants such as polyoxyalkylenated alkyl(amido) ether carboxylic acid anionic surfactants, anionic surfactants different from the abovementioned polyoxyalkylenated alkyl(amido) ether carboxylic acids, nonionic surfactants, amphoteric and zwitterionic surfactants, and mixtures thereof, and/or by using compounds conventionally present in makeup-removing compositions such as alkylpolysaccharides, fatty alcohol polyethylene glycols, oils, and mixtures thereof.

Foaming Surfactants from which the Bubbles May be Generated

As mentioned above, to generate the bubbles and/or contribute towards stabilizing them, the composition that is suitable for the invention may comprise at least one foaming surfactant such as those chosen from (i) polyoxyalkylenated alkyl(amido) ether carboxylic acid anionic surfactants, (ii) anionic surfactants different from the anionic surfactants (i), (iii) nonionic surfactants, (iv) amphoteric/zwitterionic surfactants, and mixtures thereof.

The term “anionic surfactant” means a surfactant including, as ionic or ionizable groups, only anionic groups.

In the present description, a species is termed as being “anionic” when it bears at least one permanent negative charge or when it can be ionized as a negatively charged species, under the conditions of use of the composition of the invention (for example the medium or the pH) and not comprising any cationic charge.

A species is termed as being “nonionic” when it is neither cationic nor anionic within the meaning of the present patent application, in particular when it does not include any cationic or anionic groups within the meaning of the present patent application.

The term “amphoteric/zwitterionic surfactant” or “amphoteric and zwitterionic surfactant” means a surfactant which contains central and equilibrated positive and negative charges, and which can consequently behave like an anionic or cationic surfactant by adapting to the medium in which it is present. It becomes anionic in alkaline medium and cationic in acidic medium and is equivalent to a nonionic surfactant at neutral pH.

### i) Polyoxyalkylenated Alkyl(Amido) Ether Carboxylic Acid Anionic Surfactants

A composition that is suitable for the invention may comprise at least one polyoxyalkylenated alkyl(amido) ether carboxylic acid anionic surfactant.

The polyoxyalkylenated alkyl(amido) ether carboxylic acids that may be used are chosen from those of formula (1):

R1(OC₂H₄)nOCH₂COOA (1)

in which:
R1 represents a linear or branched C₆-C₂₄ alkyl or alkenyl group, an alkyl(C₈-C₉)phenyl group, a group R2CONH—CH₂—CH₂— with R2 representing a linear or branched C₉-C₂₁ alkyl or alkenyl group; preferably, R1 is a C₈-C₂₀ alkyl group and more preferentially a C₈-C₁₈ alkyl group,
n is an integer or decimal number (mean value) ranging from 2 to 24 and preferably from 2 to 10, and
A represents a hydrogen atom, ammonium, Na, K, Li, Mg, Ca or a monoethanolamine or triethanolamine residue.

It is also possible to use mixtures of compounds of formula (1), in particular mixtures of compounds in which the groups R1 are different.

The polyoxyalkylenated alkyl(amido) ether carboxylic acids that are particularly preferred are those of formula (1) in which:
R1 represents a C₁₂-C₁₄ alkyl, cocoyl, oleyl, nonylphenyl or octylphenyl group,
A represents a hydrogen or sodium atom, and
n ranges from 2 to 20, preferably from 2 to 10.

Even more preferentially, in the compounds of formula (1), R1 represents a C₁₂ alkyl group, A represents a hydrogen or sodium atom and n ranges from 2 to 10.

Use is preferably made of polyoxyalkylenated (C₆-C₂₄)alkyl ether carboxylic acids and salts thereof, polyoxyalkylenated (C₆-C₂₄)alkylamido ether carboxylic acids, in particular those comprising from 2 to 15 alkylene oxide groups, salts thereof, and mixtures thereof.

When the anionic surfactant is in salt form, said salt may be chosen from alkali metal salts, such as the sodium or potassium salt, ammonium salts, amine salts and in particular amino alcohol salts, and alkaline-earth metal salts, such as the magnesium salt.

Examples of amino alcohol salts that may be mentioned include monoethanolamine, diethanolamine or triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular sodium or magnesium salts are preferentially used.

Among the commercial products that may preferably be used are the products sold by the company Kao under the following names:
Akypo® NP 70 (R1=nonylphenyl, n=7, A=H),
Akypo® NP 40 (R1=nonylphenyl, n=4, A=H),
Akypo® OP 40 (R1=octylphenyl, n=4, A=H),
Akypo® OP 80 (R1=octylphenyl, n=8, A=H),
Akypo® OP 190 (R1=octylphenyl, n=19, A=H),
Akypo® RLM 38 (R1=(C₁₂-C₁₄)alkyl, n=4, A=H),
Akypo® RLM 38 NV (R1=(C₁₂-C₁₄)alkyl, n=4, A=Na),
Akypo® RLM 45 CA (R1=(C₁₂-C₁₄)alkyl, n=4.5, A=H)
Akypo® RLM 45 NV (R1=(C₁₂-C₁₄)alkyl, n=4.5, A=Na),
Akypo® RLM 100 (R1=(C₁₂-C₁₄)alkyl, n=10, A=H)
Akypo® RLM 100 NV (R1=(C₁₂-C₁₄)alkyl, n=10, A=Na),
Akypo® RLM 130 (R1=(C₁₂-C₁₄)alkyl, n=13, A=H)
Akypo® RLM 160 NV (R1=(C₁₂-C₁₄)alkyl, n=16, A=Na)
or by the company Sandoz under the following names:
Sandopan DTC-Acid (R1=(C₁₃)alkyl, n=6, A=H)
Sandopan DTC (R1=(C₁₃)alkyl, n=6, A=Na)
Sandopan LS 24 (R1=(C₁₂-C₁₄)alkyl, n=12, A=Na)
Sandopan JA 36 (R1=(C₁₃)alkyl, n=18, A=H),
   and more particularly the products sold under the following names:
Akypo® RLM 45 (INCI: Laureth-5 carboxylic acid)
Akypo® RLM 100
Akypo® RLM 38.

The polyoxyalkylenated alkyl(amido) ether carboxylic acid anionic surfactants (i) may be present in an amount ranging from 0.001% to 20% by weight and preferably ranging from 0.1% to 10% by weight relative to the total weight of the composition.

(ii) Anionic Surfactants Other than the Polyoxyalkylenated Alkyl(Amido) Ether Carboxylic Acid Anionic Surfactants (i)

A composition that is suitable for the invention may comprise at least one anionic surfactant (ii) other than the polyoxyalkylenated alkyl(amido) ether carboxylic acid anionic surfactants (i).

Said anionic surfactants (ii) may comprise in their structure one or more sulfate and/or sulfonate and/or phosphate and/or carboxylate groups, and/or mixtures of these groups, preferably sulfate groups.

Said anionic surfactant(s) (ii) may be oxyethylenated and/or oxypropylenated. The total mean number of ethylene oxide (EO) and/or propylene oxide (PO) groups may then range from 1 to 50 and in particular from 1 to 10.

The carboxylic anionic surfactants that may be used may thus include at least one carboxylic or carboxylate function.

They may be chosen from the following compounds: acylglycinates, acyllactylates, acylsarcosinates, acylglutamates, alkyl-D-galactoside-uronic acids, and also the salts of these compounds; the alkyl and/or acyl groups of these compounds including from 6 to 30 carbon atoms, in particular from 12 to 28, better still from 14 to 24 or even from 16 to 22 carbon atoms; these compounds possibly being polyoxyalkylenated, in particular polyoxyethylenated, and then preferably comprise from 1 to 50 ethylene oxide units, better still from 1 to 10 ethylene oxide units.

Use may also be made of C₆-C₂₄ alkyl monoesters of polyglycoside-polycarboxylic acids, such as C₆-C₂₄ alkyl polyglycoside-citrates, C₆-C₂₄ alkyl polyglycoside-tartrates and C₆-C₂₄ alkyl polyglycoside-sulfosuccinates, and salts thereof.

Preferentially, said carboxylic anionic surfactants are chosen from:
acylglutamates, notably of C₆-C₂₄ or even C₁₂-C₂₀, such as stearoylglutamates, and in particular disodium stearoylglutamate;
acylsarcosinates, notably of C₆-C₂₄ or even C₁₂-C₂₀, such as palmitoylsarcosinates, and in particular sodium palmitoylsarcosinate;
acyllactylates, notably of C₁₂-C₂₈ or even C₁₄-C₂₄, such as behenoyllactylates, and in particular sodium behenoyllactylate;
C₆-C₂₄ and notably C₁₂-C₂₀ acylglycinates;
   in particular in the form of alkali metal, alkaline-earth metal, ammonium or amino alcohol salts; and
mixtures thereof.

The sulfonate anionic surfactants that may be used may thus include at least one sulfonate function.

They may be chosen from the following compounds: alkylsulfonates, alkylamidesulfonates, alkylarylsulfonates, olefin sulfonates, paraffin sulfonates, alkylsulfosuccinates, alkyl ether sulfosuccinates, alkylamide sulfosuccinates, alkylsulfoacetates, N-acyltaurates, acylisethionates; alkylsulfolaurates; and also the salts of these compounds; the alkyl groups of these compounds including from 6 to 30 carbon atoms, notably from 12 to 28, better still from 14 to 24 or even from 16 to 22 carbon atoms; the aryl group preferably representing a phenyl or benzyl group; these compounds possibly being polyoxyalkylenated, in particular polyoxyethylenated, and preferably comprising from 1 to 50 ethylene oxide units, better still from 2 to 10 ethylene oxide units.

Preferentially, said sulfonate anionic surfactants are chosen from:
C₆-C₂₄ and notably C₁₂-C₂₀ alkyl sulfosuccinates, notably lauryl sulfosuccinates;
C₆-C₂₄ and notably C₁₂-C₂₀, alkyl ether sulfosuccinates;
C₆-C₂₄ and preferably C₁₂-C₁₈ acylisethionates;
   in particular in the form of alkali metal, alkaline-earth metal, ammonium or amino alcohol salts; and
mixtures thereof.

The sulfate anionic surfactants that may be used may thus comprise at least one sulfate function.

They may be chosen from the following compounds: alkyl sulfates, alkyl ether sulfates, alkylamido ether sulfates, alkylaryl polyether sulfates, monoglyceride sulfates, and the salts of these compounds; the alkyl groups of these compounds comprising from 6 to 30 carbon atoms, in particular from 12 to 28, better still from 14 to 24, or even from 16 to 22 carbon atoms; the aryl group preferably representing a phenyl or benzyl group; these compounds possibly being polyoxyalkylenated, in particular polyoxyethylenated, and preferably comprising from 1 to 50 ethylene oxide units, better still from 2 to 10 ethylene oxide units. Preferentially, said sulfate anionic surfactants are chosen from:
C₆-C₂₄ and notably C₁₂-C₂₀ alkyl sulfates;
alkyl ether sulfates, notably C₆-C₂₄ or even C₁₂-C₂₀ alkyl ether sulfates, preferably comprising from 2 to 20 ethylene oxide units;
   in particular in the form of alkali metal, alkaline-earth metal, ammonium or amino alcohol salts; and
mixtures thereof.

When the anionic surfactant (ii) is in salt form, said salt may be chosen from alkali metal salts, such as sodium salts or potassium salts, ammonium salts, amine salts and in particular amino alcohol salts, and alkaline-earth metal salts, such as magnesium salts or calcium salts.

Examples of amino alcohol salts that may notably be mentioned include monoethanolamine, diethanolamine or triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts. Alkali metal or alkaline-earth metal salts and in particular sodium or magnesium salts are preferentially used.

Preferentially, said additional anionic surfactants (ii) are chosen from:
C₆-C₂₄ and notably C₁₂-C₂₀ alkyl sulfates;
C₆-C₂₄ and notably C₁₂-C₂₀ alkyl ether sulfates, preferably comprising from 2 to 20 ethylene oxide units;
C₆-C₂₄ and notably C₁₂-C₂₀ alkyl sulfosuccinates, in particular lauryl sulfosuccinates;
C₆-C₂₄ and notably C₁₂-C₂₀, alkyl ether sulfosuccinates;
C₆-C₂₄ and preferably C₁₂-C₁₈ acylisethionates;
C₆-C₂₄ and notably C₁₂-C₂₀ acylsarcosinates, in particular palmitoyl sarcosinates;
C₆-C₂₄ and notably C₁₂-C₂₀ acylglutamates;
C₆-C₂₄, in particular C₁₂-C₂₀, acylglycinates;
   in particular in the form of alkali metal, alkaline-earth metal, ammonium or amino alcohol salts; and
mixtures thereof.

Among the anionic surfactants (ii), one or more sulfate anionic surfactants are particularly preferred.

Preferably, the anionic surfactant(s) (ii) are in the form of salts, and in particular alkaline salts, in particular sodium salts, ammonium salts, amine salts, including amino alcohol salts, and/or magnesium salts. These salts preferably comprise from 2 to 5 ethylene oxide groups.

The anionic surfactants (ii) are preferably chosen from C₈-C₁₄ alkyl sulfates and/or C₈-C₁₄ alkyl ether sulfates, more preferentially from C₁₂-C₁₄ alkyl sulfates and/or C₁₂-C₁₄ alkyl ether sulfates, better still lauryl (ether) sulfates.

Sodium, triethanolamine, magnesium or ammonium C₁₂-C₁₄ alkyl sulfates and/or sodium, ammonium or magnesium C₁₂-C₁₄ alkyl ether sulfates, which are oxyethylenated, for example with 1 to 10 mol of ethylene oxide, are more preferentially used.

Better still, the anionic surfactant(s) (ii) are chosen from sodium, ammonium or magnesium C₁₂-C₁₄ alkyl ether sulfates, oxyethylenated with 2.2 mol of ethylene oxide, such as those sold, for example, under the name Texapon N702 by the company Cognis or under the name Zetesol™ 270/N-RSPO-MB by the company Zschimmer & Schwarz (sodium laureth sulfate).

The anionic surfactants (ii) different from the polyoxyalkylenated alkyl(amido) ether carboxylic acid anionic surfactants (i) may be present in an amount ranging from 0.001% to 20% by weight and preferably ranging from 0.1% to 10% by weight relative to the total weight of the composition.

(iii) Nonionic Surfactants

A composition that is suitable for the invention may comprise at least one nonionic surfactant.

The nonionic surfactants (iii) may be chosen from the following compounds:
(a) saturated or unsaturated, linear or branched, oxyethylenated alcohols comprising at least one C₈-C₄₀ alkyl chain, and comprising from 1 to 100 mol of ethylene oxide, preferably from 2 to 50 and more particularly from 2 to 40 mol of ethylene oxide and comprising one or two fatty chains;
(b) nonionic surfactants of alkyl(poly)glycoside type, notably represented by the following general formula:

R1O—(R2O)t-(G)v

in which:
R1 represents a linear or branched alkyl or alkenyl radical comprising from 6 to 24 carbon atoms and notably from 8 to 18 carbon atoms, or an alkylphenyl radical in which the linear or branched alkyl radical comprises from 6 to 24 carbon atoms and notably from 8 to 18 carbon atoms;
R2 represents an alkylene radical comprising from 2 to 4 carbon atoms;
G represents a sugar unit comprising from 5 to 6 carbon atoms;
t represents a value ranging from 0 to 10 and preferably from 0 to 4;
v represents a value ranging from 1 to 15 and preferably from 1 to 4;
(c) polyethoxylated fatty acid esters of sorbitan, preferably containing from 2 to 40 mol of ethylene oxide and comprising at least one saturated or unsaturated, linear or branched C₈-C₄₀ alkyl chain, preferably a C₁₀-C₂₈ alkyl chain (fatty acid);
(d) fatty acid esters of sucrose, preferably comprising at least one saturated or unsaturated, linear or branched C₈ to C₄₀ alkyl chain, preferably a C₁₀-C₂₈ alkyl chain (fatty acid), for instance sucrose cocoate and sucrose palmitate;
(e) polyglycerolated fatty esters, the number of glycerol groups possibly ranging from 2 to 30 and comprising at least one saturated or unsaturated, linear or branched C₈ to C₄₀ alkyl chain, preferably a C₁₀-C₂₈ alkyl chain (fatty acid), for instance polyglyceryl-5 laurate, polyglyceryl-4 laurate, polyglyceryl-10 laurate or polyglyceryl-6 dicaprate; and
(f) mixtures thereof.

Preferably, the nonionic surfactants of alkyl(poly)glycoside type are compounds of formula

R1O—(R2O)t-(G)v

in which:
R1 represents a linear or branched, saturated or unsaturated alkyl radical comprising from 8 to 18 carbon atoms;
R2 represents an alkylene radical comprising from 2 to 4 carbon atoms;
G represents a glucose, a fructose or a galactose, preferably a glucose;
t represents a value ranging from 0 to 3 and preferably from 0; and
the degree of polymerization, i.e. the value of v, possibly ranging from 1 to 15 and preferably from 1 to 4; the mean degree of polymerization more particularly being between 1 and 2.

The glucoside bonds between the sugar units are generally of 1-6 or 1-4 type and preferably of 1-4 type. Preferably, the alkyl(poly)glycoside surfactant is an alkyl(poly)glucoside surfactant. C₈ to C₁₆ alkyl(poly)glucosides of 1-4 type, and in particular decyl glucosides and caprylyl/capryl glucosides, are most particularly preferred.

Among the commercial products, mention may be made of the products sold by the company Cognis under the names Plantaren® (600 CS/U, 1200 and 2000) or Plantacare® (818, 1200 and 2000); the products sold by the company SEPPIC under the names Oramix® CG 110 and Oramix® NS 10; the products sold by the company BASF under the name Lutensol® GD 70, or the products sold by the company Chem Y under the name AG10 LK. Preferably, use is made of C₈ to C₁₆ alkyl(poly)glycosides of 1-4 type, in particular as an aqueous 53% solution, such as those sold by Cognis under the reference Plantacare® 818 UP.

According to a preferred embodiment, the nonionic surfactants (iii) may be chosen from the following compounds:
saturated or unsaturated, linear or branched, oxyethylenated C₈ to C₄₀ and in particular fatty alcohols, comprising from 1 to 100 mol of ethylene oxide, preferably from 2 to 50 mol, and more particularly of C₈ to C₂₀ and better still C₁₀ to C₁₈ comprising from 1 to 100 mol of ethylene oxide, preferably from 2 to 50 mol of ethylene oxide, more particularly from 2 to 40 mol of ethylene oxide or even from 3 to 20 mol of ethylene oxide, notably lauryl alcohol containing 4 mol of ethylene oxide (INCI name: Laureth-4) and lauryl alcohol containing 12 mol of ethylene oxide (INCI name: Laureth-12);
C₆-C₂₄ and more particularly C₈-C₁₈ alkyl(poly)glycosides; and
mixtures thereof.

According to a more preferred embodiment, the nonionic surfactants (iii) are chosen from oxyethylenated alcohols comprising at least one C₈-C₂₀ and preferably C₁₀-C₁₈ alkyl chain, comprising from 2 to 50 and in particular from 3 to 20 mol of ethylene oxide.

The nonionic surfactants (iii) may be present in an amount ranging from 0.001% to 20% by weight, preferably ranging from 0.1% to 10% by weight, relative to the total weight of the composition.

### (iv) Amphoteric/Zwitterionic Surfactants

A composition that is suitable for the invention may comprise at least one amphoteric or zwitterionic surfactant.

The amphoteric/zwitterionic surfactant(s) are non-silicone surfactants. They may notably be optionally quaternized secondary or tertiary aliphatic amine derivatives, in which the aliphatic group is a linear or branched chain comprising from 8 to 22 carbon atoms, said amine derivatives containing at least one anionic group, for example a carboxylate, sulfonate, sulfate, phosphate or phosphonate group.

Mention may notably be made of C₈-C₂₀ alkylbetaines, C₈-C₂₀ alkylsulfobetaines, (C₈-C₂₀)alkylamido(C₃-C₈)alkylbetaines and (C₈-C₂₀)alkylamido(C₆-C₅)alkylsulfobetaines.

Among the optionally quaternized secondary or tertiary aliphatic amine derivatives that may be used, as defined above. Mention may also be made of the compounds having the formulae (II) and (III) below:

Ra-CONHCH₂CH₂—N+(Rb)(Rc)-CH₂COO—,M⁺,X⁻ (II)

in which:
Ra represents a C₁₀ to C₃₀ alkyl or alkenyl group derived from an acid RaCOOH preferably present in hydrolysed coconut oil, or a heptyl, nonyl or undecyl group;
Rb represents a β-hydroxyethyl group;
Rc represents a carboxymethyl group;
M⁺ represents a cationic counterion derived from an alkali metal or alkaline-earth metal, such as sodium, an ammonium ion or an ion derived from an organic amine; and
X⁻ represents an organic or mineral anionic counterion, such as those chosen from C₁-C₄ alkyl halides, acetates, phosphates, nitrates or sulfates, (C₁-C₄)alkyl- or (C₁-C₄)alkylarylsulfonates, in particular methyl sulfate and ethyl sulfate; or alternatively M⁺ and X⁻ are absent;

Ra′-CONHCH₂CH₂—N(B)(B′) (III)

in which:
B represents the group —CH₂CH₂OX′;
B′ represents the group —(CH₂)zY′, with z=1 or 2;
X′ represents the group —CH₂COOH, —CH₂—COOZ′, —CH₂CH₂COOH or CH₂CH₂—COOZ′, or a hydrogen atom;
Y′ represents the group —COOH, —COOZ′ or —CH₂—CH(OH)SO₃H or the group —CH₂CH(OH)SO₃—Z′;
Z′ represents a cationic counterion derived from an alkali metal or alkaline-earth metal, such as sodium, an ammonium ion or an ion derived from an organic amine; and
Ra′ represents a C₁₀ to C₃₀ alkyl or alkenyl group of an acid Ra′-COOH, which is preferably present in coconut oil or in hydrolysed linseed oil, or an alkyl group, notably a C₁₇ group and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the name Miranol® C₂M Concentrate.

Use may also be made of compounds of formula (IV):

Ra″-NHCH(Y″)—(CH₂)ₙCONH(CH₂)ₙ′—N(Rd)(Re) (IV)

in which:
Y″ represents the group —COOH, —COOZ″ or —CH₂—CH(OH)SO₃H or the group CH₂CH(OH)SO₃—Z″;
Rd and Re represent, independently of each other, a C₁ to C₄ alkyl or hydroxyalkyl radical;
Z″ represents a cationic counterion derived from an alkali metal or alkaline-earth metal, such as sodium, an ammonium ion or an ion derived from an organic amine;
Ra″ represents a C₁₀ to C₃₀ alkyl or alkenyl group of an acid Ra″-COOH which is preferably present in coconut oil or in hydrolysed linseed oil; and
n and n′ represent, independently of each other, an integer ranging from 1 to 3.

Among the compounds of formula (II), mention may be made of the compound classified in the CTFA dictionary, 5th edition, 1993, under the name sodium diethylaminopropyl cocoaspartamide and sold by Chimex under the name Chimexane HB.

These compounds may be used alone or as mixtures.

Among the amphoteric/zwitterionic surfactants, use is preferentially made of (C₈-C₂₀)alkylbetaines such as cocoylbetaine, notably the product sold under the name Chegina CC-MB by the company PPU Chemco, (C₈-C₂₀)alkylamido(C₃-C₈)alkylbetaines such as cocamidopropylbetaine, and mixtures thereof, and the compounds of formula (IV) such as the sodium salt of diethylaminopropyl laurylaminosuccinamate (INCI name: sodium diethylaminopropyl cocoaspartamide).

Preferably, the amphoteric/zwitterionic surfactants are chosen from (C₈-C₂₀)alkylamido(C₃-C₈)alkylbetaines such as cocamidopropylbetaine.

More preferentially, the amphoteric/zwitterionic surfactants are chosen from (C₈-C₂₀)alkylbetaines such as cocoylbetaine.

The amphoteric/zwitterionic surfactants (iv) may be present in an amount ranging from 0.001% to 20% by weight, preferably ranging from 0.1% to 10% by weight, relative to the total weight of the composition.

The foaming surfactants (i), (ii), (iii) and/or (iv) that can generate and/or stabilize bubbles as defined in the present invention may be present in an amount ranging from 0.001% to 20% by weight, preferably ranging from 0.1% to 10% by weight, relative to the total weight of the composition.

The nature of the surfactant may have an influence on the mechanical properties of the bubbles and thus on the power required to make them collapse, smaller bubbles requiring less power.

Independently of the presence of bubbles, the surfactant(s) present in the composition that is suitable for the invention may also have a cleansing action.

Compounds Conventionally Present in Makeup-Removing Compositions from which the Bubbles May be Generated or which Contribute Towards Stabilizing them

As mentioned above, to generate and/or stabilize bubbles, a composition that is suitable for the invention may also comprise at least one compound usually used in makeup-removing compositions such as (a) an alkylpolysaccharide, (b) a fatty alcohol ether of polyethylene glycol, (c) an oil, and mixtures thereof.

### a) Alkylpolysaccharides

A composition that is suitable for the invention may comprise at least one alkylpolysaccharide.

Among the suitable alkylpolysaccharides, mention may notably be made of those of the general formula (I) below:

RO(CₓH₂ₓO)nZy (1)

in which
Z is a group derived from a reducing saccharide containing 5 or 6 carbon atoms or a reducing saccharide group containing 5 or 6 carbon atoms,
R is a C₆ to C₃₀ alkyl or alkenyl group,
x is 2 or 3,
n is from 1 to 10, and
y is from 1 to 10, including all the values and sub-ranges therebetween, such as 1.5 to 4.

The alkylpolysaccharides (also known as alkylpolyglycosides) that may be used in the cosmetic compositions include those that are commercially available, such as the surfactants APG®, Glucopon™ or Plantaren™ sold by Cognis Corporation, Ambler, PA., 19002. Examples of such surfactants include, but are not limited to:
APG® 225: an alkylpolyglycoside in which the alkyl group contains 8 to 10 carbon atoms.
APG® 425: an alkylpolyglycoside in which the alkyl group contains 8 to 16 carbon atoms.
APG® 625: an alkylpolyglycoside in which the alkyl group contains 12 to 16 carbon atoms.
APG® 300: an alkylpolyglycoside which is substantially the same as the above product but which has a different mean degree of polymerization.
Glucopon™ 600: an alkylpolyglycoside which is substantially the same as the above product but which has a different mean degree of polymerization.
Plantaren™ 2000: a C₈-C₁₆ alkylpolyglycoside with a mean degree of polymerization of 1.4.
Plantaren™ 1300: a C₁₂-C₁₆ alkylpolyglycoside with a mean degree of polymerization of 1.6
Plantaren™ 1200: a C₁₂-C₁₆ alkylpolyglycoside with a mean degree of polymerization of 1.4.

Other non-limiting examples comprise alkylpolyglycoside surfactant compositions such as those sold under the name Triton, Oramix or Montanov by SEPPIC, under the name AG by Kao, under the name Atlas G-73500 by Uniqema, under the name Marlosan 240 by Condea Chemie or under the name Desulf GOS-P-60WCG by DeForest Enterprises.

The alkylpolysaccharides (a) may be present in an amount ranging from 0.01% to 20% by weight, preferably ranging from 0.1% to 10% by weight, relative to the total weight of the composition.

### b) Fatty Alcohol Ethers of Polyethylene Glycol

A composition that is suitable for the invention may comprise at least one fatty alcohol ether of polyethylene glycol.

The suitable fatty alcohol ethers of polyethylene glycol comprise polyethylene glycol derivatives of glyceryl cocoate, of glyceryl caproate, of glyceryl caprylate, of glyceryl tallow, of glyceryl palmate, of glyceryl stearate, of glyceryl laurate, of glyceryl oleate, of glyceryl ricinoleate and of glyceryl fatty esters derived from triglycerides, such as palm oil, almond oil, olive oil, corn oil, and mixtures thereof.

These fatty alcohol ethers of polyethylene glycols (b) may be used in an amount ranging from 0.01% to 25% by weight and preferably ranging from 0.1% to 10% by weight relative to the total weight of the composition, and are preferably surfactants.

### c) Oils

A composition that is suitable for the invention may comprise at least one oil.

The suitable oils comprise those commonly used in makeup removers. These oils may be chosen from mineral oils, plant oils, synthetic oils and silicone oils, and mixtures thereof. Among the mineral oils that can constitute the oily phase, mention may notably be made of liquid paraffin or a mineral oil and higher aliphatic hydrocarbons, for instance isohexadecane. Among the plant oils, mention may notably be made of jojoba oil, meadowfoam seed oil, apricot kernel oil and also safflower oil. Among the silicone oils, mention may notably be made of the cyclopentadimethylsiloxane sold under the name Volatile Silicone 7158 by the company Union Carbide or DM Fluid 0.6 cs, a linear volatile silicone oil sold by Shin-Etsu or the polydimethylsiloxanes sold under the name DC200 by Dow Corning.

Among the synthetic products, mention may notably be made of esters such as alkyl palmitates in which the alkyl radical contains from 2 to 10 carbon atoms, such as isopropyl palmitate or 2-ethylhexyl palmitate, and alkyl adipates in which the alkyl radical contains from 2 to 10 carbon atoms, such as bis(2-ethylhexyl) adipate, or esters such as isononyl isononanoate or derivatives such as meadowfoam.

These oils (c) may be used in amounts ranging from 0.1% to 100% by weight, preferably ranging from 0.1% to 60% by weight, relative to the total weight of the composition.

As other examples, a compound with stabilizing action that is suitable for the invention is preferably chosen from:
fatty alcohols;
polyalkylene glycols;
fatty acid esters of polyglycerol;
polyalkylene glycol ethers of alkylglucose;
celluloses;
crosslinked polymers formed by the reaction of a C₁₀-C₁₈ alkylglucoside with 1,3-dichloro-2-propanol, and sulfonated with 3-chloro-2-hydroxypropyl sulfonate;
fatty acid alkanolamides;
and mixtures thereof.

A) The fatty alcohols are preferably those including a saturated linear alkyl chain containing from 10 to 18 carbon atoms, for instance lauryl alcohol, myristyl alcohol, cetyl alcohol, stearyl alcohol and mixtures thereof such as mixtures of cetyl alcohol and stearyl alcohol (cetearyl alcohol);

B) The polyalkylene glycols are preferably those of formula (II)

H—[O—R-]n-OH (II)

in which
R represents a linear alkyl chain containing from 1 to 4 carbon atoms, and
n is an integer ranging from 4 to 100 000 and advantageously from 4 to 50 000.

According to a particular embodiment of the invention, a polyalkylene glycol in accordance with the invention is a polyethylene glycol.

According to a preferred embodiment, a polyethylene glycol in accordance with the invention may be chosen from PEG-8, such as the product sold under the trade name Polyethylene Glycol 400 DUB® by the company Clariant, or PEG-45M such as the product sold under the trade name Polyox WSR N 60 KR by the company Dow Chemical.

C) The polyglyceryl fatty acid esters are preferably monoesters of a fatty acid including a saturated alkyl chain containing from 10 to 18 carbon atoms and of a polyglycerol containing from 2 to 30 mol of glycerol groups.

According to a preferred embodiment, a polyglycerolated fatty acid ester in accordance with the invention may be chosen from polyglyceryl-2 laurate, such as the product sold under the trade name SunSoft Q-12D-Co by the company Taiyo Kagaku.

D) Polyalkylene glycol ethers of alkylglucose, and preferably poly(C₁-C₄)alkylene glycol ethers of (C₁-C₄)alkylglucose, in particular methyl gluceth-10, such as the product sold under the trade name Glucam E-10 Humectant® by the company Lubrizol and methyl gluceth-20, such as the product sold under the trade name Glucam E-20 Humectant® by the company Lubrizol.

E) The celluloses are preferably polyalkylene glycol ethers of alkylcellulose, for instance hydroxypropyl methylcellulose such as the product sold under the trade name Benecel K100M Hydroxypropylmethyl Cellulose® by the company Ashland or the product sold under the name Methocel™ F4M Personal Care Grade by the company Dow.

F) The crosslinked polymers formed by the reaction of a C₁₀-C₁₈ alkylglucoside with 1,3-dichloro-2-propanol, and sulfonated with 3-chloro-2-hydroxypropyl sulfonate are preferably chosen from the polymers having the respective INCI names:
Sodium Hydroxypropylsulfonate Laurylglucoside Crosspolymer sold under the trade name Poly Suganate 160PR by the company Colonial Chemical Inc.;
Sodium Hydroxypropylsulfonate Cocoglucoside Crosspolymer sold under the trade name Poly Suganate 124PR by the company Colonial Chemical Inc.; and
Sodium Hydroxypropylsulfonate Decylglucoside Crosspolymer sold under the trade name Poly Suganate 100Po by the company Colonial Chemical Inc.

G) The fatty acid alkanolamides are preferably chosen from fatty acid C₁₂-C₁₈ alkanolamides such as Cocamide MEA such as the product sold under the trade name Comperlan CMEA® by the company BASF.

Among the list of compounds A) to G) with a stabilizing action which are suitable for the invention, mention may advantageously be made of polyethylene glycols and polyglycerolated fatty acid esters, more particularly chosen from PEG-45M and Polyglyceryl-2 Laurate.

Preferentially, among this list of compounds A) to G) with stabilizing action which are suitable for the invention, use may be made of celluloses, in particular polyalkylene glycol ethers of alkylcellulose such as hydroxypropyl methylcellulose.

Advantageously, a cosmetic composition that is suitable for the invention comprises an amount of compounds with stabilizing action ranging from 0.001% to 20% by weight, preferably ranging from 0.01% to 10% by weight, relative to the total weight of the composition.

Even more advantageously, when the compound with stabilizing action is a cellulose, in particular polyalkylene glycol ethers of alkylcellulose such as hydroxypropyl methylcellulose, this compound may be present in the composition in an amount ranging from 0.05% to 1% by weight, preferably from 0.1% to 0.9% by weight and more preferentially 0.5% by weight, relative to the total weight of the composition.

According to a particular embodiment, the composition that is suitable for the invention comprises as compound with stabilizing action at least one cellulose, in particular chosen from polyalkylene glycol ethers of alkylcellulose, preferably hydroxypropyl methylcellulose. According to another particular embodiment, the composition that is suitable for the invention comprises as compound with stabilizing action at least one anionic surfactant (ii) such as those defined in the present text, in particular chosen from sodium, ammonium or magnesium C₁₂-C₁₄ alkyl ether sulfates, oxyethylenated with 2.2 mol of ethylene oxide, preferably sodium laureth sulfate.

According to another particular embodiment, the composition that is suitable for the invention comprises as compound with stabilizing action at least one amphoteric/zwitterionic surfactant (iv) such as those defined in the present text, in particular chosen from (C₈-C₂₀)alkylbetaines, preferably cocoylbetaine.

### Cationic Foaming Surfactant

According to one embodiment, the composition according to the invention may comprise at least one cationic surfactant, in particular in the case where it comprises an amphoteric foaming surfactant. The cationic agents used may also have a thickening role which is favourable towards bubble stabilization.

The cationic surfactants that may be used according to the present invention are notably salts of optionally polyoxyalkylenated primary, secondary or tertiary fatty amines, quaternary ammonium salts, imidazoline derivatives and amine oxides of cationic nature, and mixtures thereof.

Examples of quaternary ammonium salts include:
those that have the general formula (IV) below:

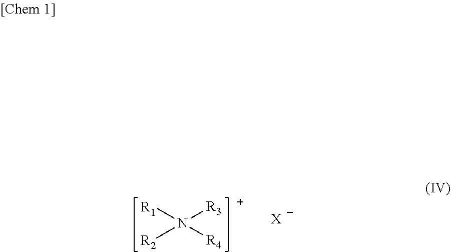

in which the radicals R₁ to R₄, which may be identical or different, represent a linear or branched aliphatic radical including from 1 to 30 carbon atoms, or an aromatic radical such as aryl or alkylaryl. The aliphatic radicals may include heteroatoms notably such as oxygen, nitrogen, sulfur and halogens. The aliphatic radicals are chosen, for example, from alkyl, alkoxy, polyoxy(C₂-C₆)alkylene, alkylamide, (C₁₂-C₂₂)alkylamido(C₂-C₆)alkyl, (C₁₂-C₂₂)alkylacetate and hydroxyalkyl radicals, including from about 1 to 30 carbon atoms; X is an anion chosen from the group of halides, phosphates, acetates, lactates, (C₂-C₆)alkyl sulfates and alkyl or alkylaryl sulfonates. Preferably, R₁ and R₂ denote a C₁-C₄ alkyl or a C₁-C₄ hydroxyalkyl.
quaternary ammonium salts of imidazolinium, for instance the salt of formula (V) below:

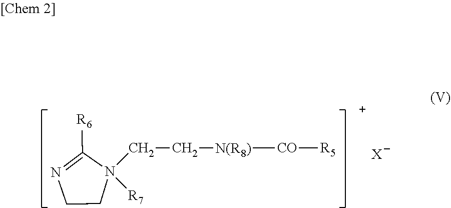

in which R₅ represents an alkenyl or alkyl radical including from 8 to 30 carbon atoms, for example coconut kernel fatty acid derivatives, R₆ represents a hydrogen atom, a C₁-C₄ alkyl radical or an alkenyl or alkyl radical including from 8 to 30 carbon atoms, R₇ represents a C₁-C₄ alkyl radical, R₈ represents a hydrogen atom or a C₁-C₄ alkyl radical and X⁻ is an anion chosen from the group of halides, phosphates, acetates, lactates, alkyl sulfates, and alkyl or alkylaryl sulfonates. Preferably, R₅ and R₆ denote a mixture of alkenyl or alkyl radicals including from 12 to 21 carbon atoms, for example tallow fatty acid derivatives, R₇ denotes methyl and R₈ denotes hydrogen.
the diquaternary ammonium salts of formula (VI):

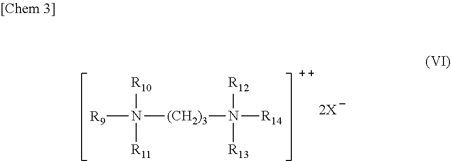

in which R₉ denotes an aliphatic radical including from about 16 to 30 carbon atoms, R₁₀, R₁₁, R₁₂, R₁₃ and R₁₄, which may be identical or different, are chosen from hydrogen or an alkyl radical including from 1 to 4 carbon atoms, and X is an anion chosen from the group of halides, acetates, phosphates, nitrates and methyl sulfates.
quaternary ammonium salts comprising at least one ester function, for example those of formula (VII) below:

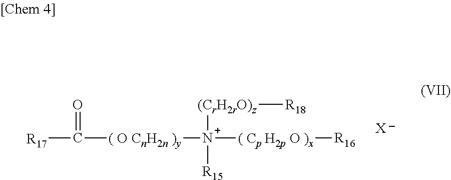

in which:
R₁₅ is chosen from C₁-C₆ alkyl radicals and C₁-C₆ hydroxyalkyl or dihydroxyalkyl radicals;
R₁₆ is chosen from:
   the radical R₁₉—C(═O)—,
   linear or branched, saturated or unsaturated C₁-C₂₂ hydrocarbon-based radicals R₂₀,
   a hydrogen atom,
R₁₈ is chosen from:
   the radical R₂₁—C(═O)—,
   linear or branched, saturated or unsaturated C₁-C₆ hydrocarbon-based radicals R₂₂,
   a hydrogen atom,
R₁₇, R₁₉ and R₂₁, which may be identical or different, are chosen from linear or branched, saturated or unsaturated C₇-C₂₁ hydrocarbon-based radicals;
n, p and r, which may be identical or different, are integers ranging from 2 to 6;
y is an integer ranging from 1 to 10;
x and z, which may be identical or different, are integers ranging from 0 to 10; and
X⁻ is a simple or complex and organic or mineral anion;
with the proviso that the sum x+y+z is from 1 to 15, that when x is 0 then R₁₆ denotes R₂₀, and that when z is 0 then R₁₈ denotes R₂₂.

The alkyl radicals R₁₅ may be linear or branched, and more particularly linear.

Preferably, R₁₅ denotes a methyl, ethyl, hydroxyethyl or dihydroxypropyl radical, and more particularly a methyl or ethyl radical.

Advantageously, the sum x+y+z is from 1 to 10.

When R₁₆ is a hydrocarbon-based radical R₂₀, it may be long and contain from 12 to 22 carbon atoms, or short and contain from 1 to 3 carbon atoms.

When R₁₈ is a hydrocarbon-based radical R₂₂, it preferably contains 1 to 3 carbon atoms.

Advantageously, R₁₇, R₁₉ and R₂₁, which may be identical or different, are chosen from linear or branched, saturated or unsaturated C₁₁-C₂₁ hydrocarbon-based radicals, and more particularly from linear or branched, saturated or unsaturated C₁₁-C₂₁ alkyl and alkenyl radicals.

Preferably, x and z, which may be identical or different, are equal to 0 or 1.

Advantageously, y is equal to 1.

n, p and r, which may be identical or different, are preferably 2 or 3 and even more particularly are equal to 2.

The anion is preferably a halide (chloride, bromide or iodide) or an alkyl sulfate, more particularly methyl sulfate. However, use may be made of methanesulfonate, phosphate, nitrate, tosylate, an anion derived from an organic acid, such as acetate or lactate, or any other anion that is compatible with the ammonium bearing an ester function.

The anion X⁻ is even more particularly chloride or methyl sulfate.

Use is made more particularly of the ammonium salts of formula (VII) in which:
R₁₅ denotes a methyl or ethyl radical;
x and y are equal to 1;
z is equal to 0 or 1;
n, p and r are equal to 2;
R₁₆ is chosen from:
   the radical R₁₉—C(═O)—;
   methyl, ethyl or C₁₄-C₂₂ hydrocarbon-based radicals;
   a hydrogen atom;
R₁₈ is chosen from:
   the radical R₂₁—C(═O)—;
   a hydrogen atom;
R₁₇, R₁₉ and R₂₁, which may be identical or different, are chosen from linear or branched, saturated or unsaturated C₁₃-C₁₇ hydrocarbon-based radicals and preferably from linear or branched, saturated or unsaturated C₁₃-C₁₇ alkyl and alkenyl radicals.

Advantageously, the hydrocarbon-based radicals are linear.

Among the quaternary ammonium salts of formula (IV), preference is given, on the one hand, to tetraalkylammonium chlorides, for instance dialkyldimethylammonium or alkyltrimethylammonium chlorides in which the alkyl radical includes from about 12 to 22 carbon atoms, in particular behenyltrimethylammonium chloride, distearyldimethylammonium chloride, cetyltrimethylammonium chloride and benzyldimethylstearylammonium chloride, or else, on the other hand, to palmitylamidopropyltrimethylammonium chloride or the stearamidopropyldimethyl(myristyl acetate)ammonium chloride sold under the name Ceraphyl® 70 by the company Van Dyk.

Examples of compounds of formula (V) that may be mentioned include the diacyloxyethyldimethylammonium, diacyloxyethylhydroxyethylmethylammonium, monoacyloxyethyldihydroxyethylmethylammonium, triacyloxyethylmethylammonium and monoacyloxyethylhydroxyethyldimethylammonium salts (notably chloride or methyl sulfate), and mixtures thereof. The acyl radicals preferably contain 14 to 18 carbon atoms and are more particularly derived from a plant oil, for instance palm oil or sunflower oil. When the compound contains several acyl radicals, these radicals may be identical or different.

These products are obtained, for example, by direct esterification of triethanolamine, triisopropanolamine, an alkyldiethanolamine or an alkyldiisopropanolamine, which are optionally oxyalkylenated, with fatty acids or with fatty acid mixtures of plant or animal origin, or by transesterification of the methyl esters thereof. This esterification is followed by a quaternization using an alkylating agent such as an alkyl halide (preferably a methyl or ethyl halide), a dialkyl sulfate (preferably dimethyl or diethyl sulfate), methyl methanesulfonate, methyl para-toluenesulfonate, glycol chlorohydrin or glycerol chlorohydrin.

Such compounds are sold, for example, under the names Dehyquart® by the company Cognis, Stepanquat® by the company Stepan, Noxamium by the company Ceca, and Rewoquat® WE 18 and Rewoquat® W75 by the company Degussa.

Use may also be made of the ammonium salts containing at least one ester function that are described in patents U.S. Pat. Nos. 4,874,554 and 4,137,180.

Quaternary diammonium salts of formula (VI) that are suitable for use in the invention notably comprise propanetallowdiammonium dichloride.

The cationic surfactants are generally present in the composition according to the invention in an amount ranging from 0.01% to 10% by weight, preferably ranging from 0.1% to 1% by weight relative to the total weight of the composition.

### Soap

According to another particular embodiment, the composition that is suitable for the invention comprises at least one soap.

The soap used in the context of the present invention is an organic soap of a fatty acid containing from 10 to 22 carbon atoms, more preferentially from 12 to 18 carbon atoms.

The fatty acid that is suitable for the present invention may be chosen from linear fatty acids, branched fatty acids and mixtures thereof.

The fatty acid may notably be chosen from caproic acid, capric acid, caprylic acid, oleic acid, linoleic acid, lauric acid, myristic acid, stearic acid and palmitic acid, and mixtures thereof.

Preferably, the fatty acid is a linear fatty acid.

According to a preferred embodiment, the fatty acid may be chosen from lauric acid, myristic acid, stearic acid and mixtures thereof.

A neutralizer may be added so as to neutralize the soap.

The neutralizer may be chosen from amino alcohols such as ethanolamine, amino sugars, amino acids and the alkaline salts thereof. The neutralizer that is most preferred is triethanolamine.

Neutralization of the soap may be obtained by having a mole ratio between the neutralizer and the fatty acid of at least 1:1.43, preferably of at least 1:1.25.

According to another embodiment, the mole ratio between the neutralizer and the fatty acid ranges from 1:1.43 to 1:1, in particular from 1:1.25 to 1:1.05.

The amount to be taken into account for the calculation of the amount of soap is the total content of fatty acids without the neutralizer.

Thus, the soap content in the composition may range, for example, from 5% to 50% by weight, more preferentially from 10% to 35% by weight and most preferably from 15% to 25% by weight relative to the total weight of the composition.

In the present invention, the weight ratio of the fatty acid(s) to the surfactant(s) may range from 1.5:1.0 to 5.0:1.0, preferably from 1.6:1.0 to 4.5:1.0, more preferably from 1.7:1.0 to 4.0:1.0.

According to a preferred embodiment, the weight ratio of the linear fatty acid(s) to the surfactant(s) may range from 1.5:1.0 to 5.0:1.0, preferably from 1.6:1.0 to 4.5:1.0, more preferably from 1.7:1.0 to 4.0:1.0.

Advantageously, the simultaneous presence of soap and of surfactant(s) gives a balance between an open foam with large bubbles and a creamier foam, in addition to rapid rinsing and a sensation of cleanliness after application of the composition.

### Additives

A composition that is suitable for the invention may also comprise a wide diversity of additives usually considered in the field of the galenical formulation of cosmetic compositions, in particular of cleansing compositions or makeup-removing compositions for human keratin materials.

Thus, the compositions that are suitable for the invention may also notably comprise as additives gelling agents, conventional hydrophilic or lipophilic thickeners, hydrophilic or lipophilic active agents, preserving agents (e.g. phenoxyethanol), antioxidants, fragrances, essential oils, emulsifiers, moisturizers, chelating agents, sequestrants (e.g. EDTA and salts thereof), vitamins, emollients, polymers different from those mentioned previously, conditioning agents, humectants, proteins, polypeptides, amino acids and derivatives thereof, buffer agents, viscosity modifiers, plant extracts or plants. Certain powders or particulate compounds may contribute towards the stability of the bubbles in the medium by being present at the gas/liquid interface, notably when these powders or particulate compounds are generated from solid constituents which dissolve gradually in the medium.

The additives are generally present in the composition according to the invention in an amount ranging from 0% to 20% by weight and preferably ranging from 0.01% to 10% by weight relative to the total weight of the composition.

These additives and the amounts thereof should be such that they do not modify the property(ies) desired for the composition of the invention.

According to one variant, a composition that is suitable for the invention may comprise an aqueous medium or aqueous phase, i.e. a medium including an amount of water ranging from 0.1% to 99% by weight, preferably ranging from 50% to 90% by weight and better still ranging from 60% to 90% by weight, relative to the total weight of the composition.

The aqueous phase of the compositions according to the invention may contain, besides water, one or more solvents chosen from monoalcohols including from 1 to 6 carbon atoms, and polyols, and mixtures thereof.

A monoalcohol that may notably be mentioned is ethanol.

When the monoalcohols are present, the amount thereof in the composition may range, for example, from 0.1% to 50% by weight, preferably from 0.5% to 15% by weight and better still from 5% to 15% by weight relative to the total weight of the composition.

For the purposes of the present invention, the term “polyol” should be understood as meaning any organic molecule including at least two free hydroxyl groups.

Polyols that may notably be mentioned include glycerol; glycols such as butylene glycol, isoprene glycol or propylene glycol, sorbitol; sugars such as glucose, fructose, maltose, lactose and sucrose; and mixtures thereof.

When the polyols are present, the amount thereof in the composition may range, for example, from 0.1% to 40% by weight, preferably from 0.5% to 15% by weight and better still from 5% to 15% by weight relative to the total weight of the composition.

According to another variant, a composition that is suitable for the invention may also be anhydrous.

For the purposes of the present invention, the term “anhydrous” refers to a composition comprising a content of less than or equal to 1% by weight and preferably less than or equal to 0.5% by weight of water relative to the total weight of said composition, or is even free of water. Where appropriate, such small amounts of water may notably be introduced by ingredients of the composition that may contain residual amounts thereof.

According to yet another variant, a composition that is suitable for the invention may also be essentially oily, i.e. it may comprise an oily phase or fatty phase, i.e. a medium including an amount of oil(s) ranging from 0.1% to 99% by weight, preferably ranging from 50% to 90% by weight and better still ranging from 60% to 90% by weight, relative to the total weight of the composition.

A person skilled in the art will take care to select the ingredients included in the composition, and also the amounts thereof, so that they do not harm the desired properties of the compositions of the present invention.

The pH of the composition, if it is aqueous, may be between 4 and 7.5, notably between 4.5 and 6 or may be 10 as is the case for certain foaming products such as soaps.

The cosmetic composition that is suitable for the invention may be rinsed off or left on after having been applied to the keratin materials and having been subjected to the acoustic waves.

A composition that is suitable for the invention may be prepared according to techniques that are well known to those skilled in the art.

A composition according to the invention may be in any presentation form conventionally used according to the envisaged applications, and notably in the form of an aqueous, alcoholic or aqueous-alcoholic or oily solution or suspension, a solution or dispersion of the lotion or serum type, an oil-in-water or water-in-oil emulsion, a microemulsion, an aqueous or anhydrous gel, or any other suitable cosmetic form.

The cosmetic composition according to the invention notably finds a particularly advantageous application in the field of body and/or hair hygiene, notably for cleansing the hair and/or the scalp, and also for cleansing and/or removing makeup from bodily and/or facial skin. It may thus constitute a shampoo or a shower gel, or alternatively a mask to be rinsed off.

The expressions “between . . . and . . . ”, and “ranging from . . . to . . . ” should be understood as meaning limits included, unless otherwise specified.

In the description and the examples, the percentages are weight percentages. The ingredients are mixed in the order and under the conditions that are readily determined by a person skilled in the art.

### BRIEF DESCRIPTION OF THE DRAWINGS

The invention may be understood more clearly on reading the following detailed description of non-limiting implementation examples thereof and on examining the appended drawing, in which:

FIG. 1 schematically illustrates the use of an example of a treatment device according to the invention,

FIG. 2 is a schematic view, in longitudinal cross section, of another example of a treatment device for performing the invention,

FIG. 3 is a similar view to FIG. 2 of an implementation variant of the invention,

FIG. 4 schematically represents various components of the device of FIG. 3, and

FIG. 5 schematically represents an implementation variant.

### DETAILED DESCRIPTION

The method according to the invention involves the exposure of bubbles present in a cosmetic composition to acoustic waves.

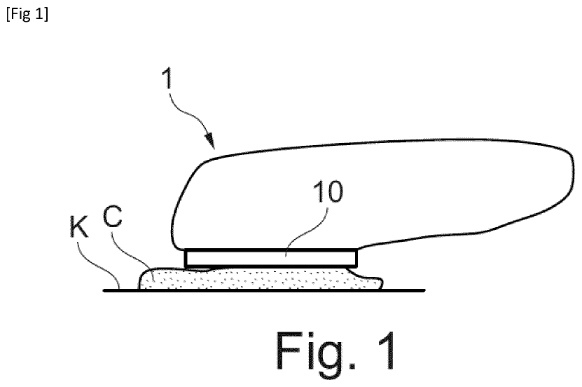

FIG. 1 illustrates a first example of implementing the invention, in which a cosmetic composition C containing bubbles is present at the surface of the keratin materials K to be treated, and a treatment device 1 is brought into contact with the composition C to emit acoustic waves therein.

The composition C is, for example, in the form of a foam.

The keratin materials K consist, for example, of facial skin or the hair.

It is a matter, for example, of cleansing the skin to remove traces of makeup more quickly and efficiently.

The treatment device 1 includes a handpiece which carries a sonotrode 10, in contact with the composition, and from which the acoustic waves are emitted.

The handpiece can be handled so as to allow some leeway with the keratin materials K and to avoid contact of the sonotrode therewith.

As a variant, the handpiece is arranged to maintain such a gap, by means of one or more members intended to contact the keratin materials and with respect to which the sonotrode 10 is set back.

Under the effect of the acoustic waves, the bubbles undergo collapse on themselves, which generates a shock wave that proves to be efficient for cleansing the skin.

### EXAMPLES

### Example 1

### Tests on Artificial Skin

The composition is, for example, one of the compositions C₁ to C₁₂ mentioned hereinbelow.

Samples of artificial skin (BioSkin brand) are prepared, on which is applied a long-lasting foundation to a thickness of greater than 20 microns.

The product is left to dry for 15 minutes at room temperature, and the drying is completed for 2 minutes with a hairdryer.

A sonotrode emitting acoustic waves at a frequency of about 35 kHz is used, and is maintained about 2 to 3 mm from the skin, in contact with the composition.

The sonotrode is moved slowly in contact with the composition (within the foam), without touching the foundation film.

Strong removal of the foundation is observed in the regions where the sonotrode passes while active, and no removal is observed in the regions where it passes while switched off.

Great difficulty in removing the makeup by simply passing over it with a sponge or a brush is moreover observed, which demonstrates the cleansing effect obtained in the invention.

### Example 2

### Tests on Natural Skin Explants

Similar tests are performed on skin explants, and removal of the foundation is observed after one or even two passes.

The composition tested is, for example, the same as that of Example 1, to within one dilution factor.

Tests of removal of indelible marker marks are also performed, and virtual disappearance of the marks is observed after two passes.

### Compositions Tested

Compositions C1 to C12 in the form of a foaming solution are prepared by mixing the compounds described in Tables 1, 2, 3 and 4 below.

The compositions are prepared using the weight proportions described below. The percentages are given on a weight basis relative to the total weight of the composition. The percentage of active materials (AM) is also indicated where necessary.

TABLE 1

| Compounds | C1 | C2 | C3 | C4 |
|---|---|---|---|---|
| Cocoylbetaine | 43.30% | 43.30% | 43.30% | 43.30% |
| (Chegina CC-MB sold by | (13.00% AM) | (13.00% AM) | (13.00% AM) | (13.00% AM) |
| the company PPU Chemco) | | | | |
| Hydroxyproprylmethylcellulose | — | 0.10% | 0.20% | 0.30% |
| (Methoceltm F4M Personal | | | | |
| Care Grade sold by | | | | |
| the company Dow) | | | | |
| Water | 56.70% | 56.60% | 56.50% | 56.40% |

TABLE 2

| Compounds | C5 | C6 |
|---|---|---|
| Cocoylbetaine | 43.30% | 43.30% |
| (Chegina CC-MB sold by the company | (13.00% AM) | (13.00% AM) |
| PPU Chemco) | | |
| Hydroxypropylmethylcellulose | 0.40% | 0.50% |
| (Methoceltm F4M Personal Care Grade | | |
| sold by the company Dow) | | |
| Water | 56.30% | 56.20% |

TABLE 3

| Compounds | C7 | C8 | C9 | C10 |
|---|---|---|---|---|
| Sodium laureth sulfate | 18.57% | 18.57% | 18.57% | 18.57% |
| (ZetesolTM 270/N - RSPO-MB | (13.00% AM) | (13.00% AM) | (13.00% AM) | (13.00% AM) |
| sold by the company | | | | |
| Zschimmer & Schwarz) | | | | |
| Hydroxypropylmethylcellulose | — | 0.10% | 0.20% | 0.30% |
| (Methoceltm F4M Personal | | | | |
| Care Grade sold by the | | | | |
| company Dow) | | | | |
| Water | 81.43% | 81.33% | 81.23% | 81.13% |

TABLE 4

| Compounds | C11 | C12 |
|---|---|---|
| Sodium laureth sulfate | 18.57% | 18.57% |
| (ZetesolTM 270/N - RSPO-MB sold by | (13.00% AM) | (13.00% AM) |
| the company Zschimmer & Schwarz) | | |
| Hydroxypropylmethylcellulose | 0.40% | 0.50% |
| (Methoceltm F4M Personal Care Grade | | |
| sold by the company Dow) | | |
| Water | 81.03% | 80.93% |

The following bubble sizes are measured as summarized in Table 5 below:

TABLE 5

| Composition | d bubble min (m) | d bubble med (m) | d bubble max (m) |
|---|---|---|---|
| C1 | 1.93E−04 | 4.21E−04 | 6.65E−04 |
| C2 | 1.71E−04 | 2.40E−04 | 3.97E−04 |
| C3 | 1.12E−04 | 1.00E−04 | 3.14E−04 |
| C4 | 9.00E−05 | 1.58E−04 | 2.43E−04 |
| C5 | 6.80E−05 | 1.39E−04 | 2.43E−04 |
| C6 | 8.60E−05 | 1.72E−04 | 2.67E−04 |
| C7 | 9.30E−05 | 1.58E−04 | 2.99E−04 |
| C8 | 9.80E−05 | 1.58E−04 | 2.20E−04 |
| C9 | 9.90E−05 | 1.42E−04 | 2.87E−04 |
| C10 | 6.40E−05 | 1.03E−04 | 1.46E−04 |
| C11 | 6.40E−05 | 9.70E−05 | 1.44E−04 |
| C12 | 5.90E−05 | 8.57E−05 | 1.38E−04 |

The compositions in which the hydroxypropyl methylcellulose polymer is present in an amount of from 0.3% to 0.5% by weight, relative to the total weight of the composition, are those which contain the smallest bubbles, and which tend to be the most efficient.

### Example 3

### Tests on Hair

Hair samples are prepared, namely:
an untreated lock serving as reference;
a lock of the same hair dyed with the product Colorista Washout L′Oreal Paris;
a lock of the same hair dyed with the dye product Majirouge 6.66 L′Oréal Pro.

A sonotrode emitting at 34 kHz is passed 10 times over each of the locks.

It is observed that the reference lock substantially conserves its colour despite the treatment.

The method produces significant lightening of the locks dyed with the composition Colorista Washout, with a delta E of the order of 9, and less decolouring, but which remains visible, of the locks treated with the composition Majirouge, with a delta E of the order of 4.

In the example of FIG. 1, composition C is applied, for example using a pressurized container, which generates a foam, and the handpiece carrying the sonotrode is then brought in contact therewith.

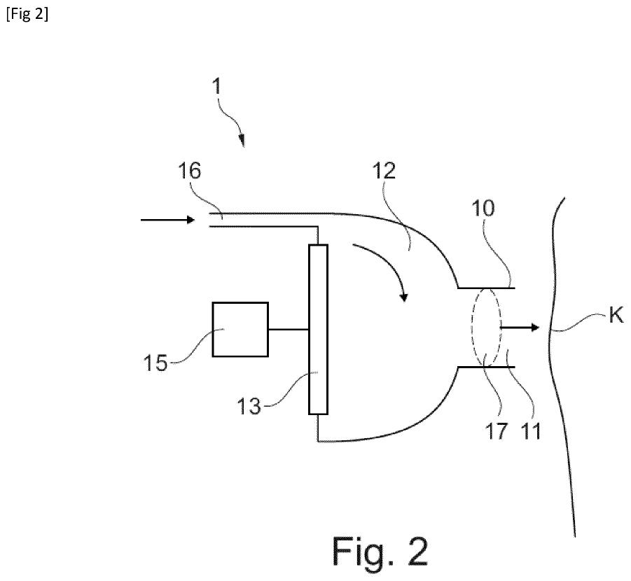

The composition may also be applied, as illustrated in FIG. 2, by the device which generates the acoustic waves.

In this figure, the device 1 includes an applicator 10 arranged to dispense composition C onto the area to be treated, for example via at least one orifice 11.

The device 1 may include, as illustrated, a chamber 12 in which composition C circulates and at least one ultrasonic transducer 13 to emit acoustic waves in the chamber 12. The transducer 13 is powered by a generator 15, which may or may not form part of the handpiece, for example being present in a base station to which the handpiece is connected via a cable.

Composition C may be brought into the chamber 12 via a pipe 16 and may come, for example, from a composition reservoir.

The device 1 includes a bubble generator 17 for generating in the composition gas bubbles which will be subjected to the acoustic waves emitted by the transducer 13.

This bubble generator 17 is only represented schematically in FIG. 2 since it may have very diverse implementation forms, involving electrolysis or other phenomena.

In one variant, the bubbles are generated in the reservoir.

The bubbles are generated a sufficiently short distance from the outlet 11 so as to come close to or in contact with the area to be treated.

Composition C may contain any compound, as detailed previously, enabling the bubbles to be generated and to exist in the composition for a time sufficient for them to be transported to the area to be treated.

In the example of FIG. 2, the handpiece is moved along the area to be treated and the composition that is dispensed via the outlet 11 is not recycled.

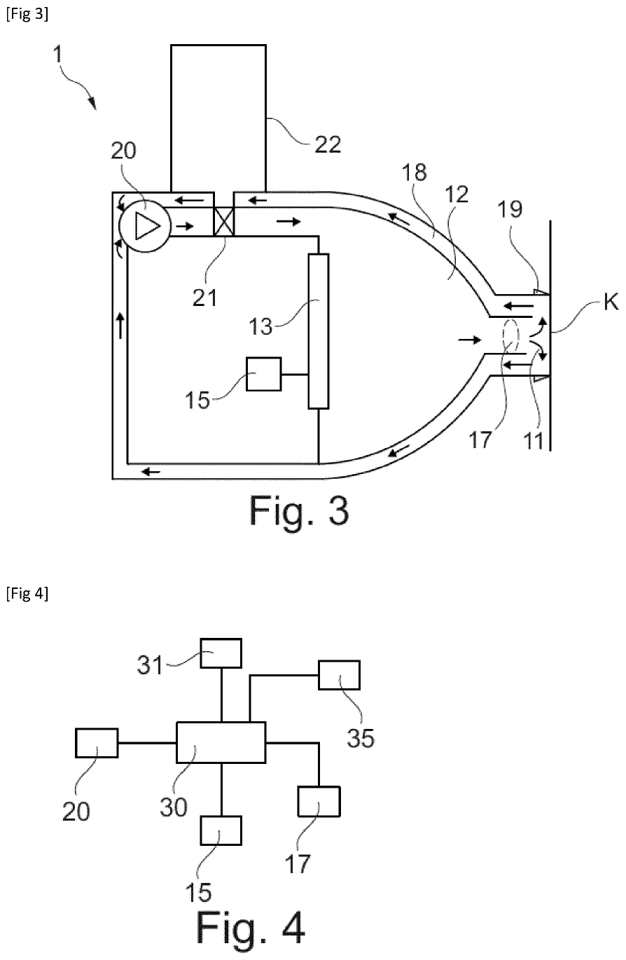

In the variant of FIG. 3, recycling of the composition is performed and represented.

In this example of FIG. 3, the device 1 used for performing the method according to the invention includes at least one transducer 13 emitting acoustic waves in a chamber 12, as in the example of FIG. 2.

However, composition C which is dispensed via the outlet 11 onto the area K to be treated is recovered by at least one pipe 18 in order to be recycled.

In the example considered, this pipe 18 emerges around the outlet 11 so as to recover the composition that has been in contact with the area to be treated.

The device 1 may include, where appropriate, around the pipe 18 a sealing member 19 such as a flexible lip to contain the composition and to facilitate its return via the pipe 18.

The pipe 18 communicates with a suction pump 20 which may, as illustrated, send the returned composition into a filter 21. Said filter may be arranged to stop, for example, the particles in suspension in the composition, for instance skin debris removed during the cleansing.

The composition is sent again at the outlet of the filter 21 into the chamber.

The composition may come from a reservoir 22 represented schematically, for example carried by the handpiece.

This reservoir makes it possible to fill the circuit in which the composition circulates during the functioning of the device, and to compensate for any losses of composition, in the event that a portion thereof is not recycled.

The filter 21 may be carried by the reservoir, for example so as to allow its automatic replacement when the composition is used up and when the reservoir is replaced.

As illustrated in FIG. 4, the device 1 may include an electronic circuit governing the functioning of the transducer(s) 13, including a control unit 30, for example with a microcontroller, carried by the handpiece and/or divided between a possible base station and the handpiece, or present only on the base station.

This control unit 30 can communicate with a man-machine interface 31, which may include a screen and/or control buttons, or even communicate via a wireless connection with a terminal such as a cell phone.

The interface 31 may make it possible to adjust, for example, certain operating parameters of the device, for example the intensity with which the acoustic waves are emitted and/or the bubbles generated.

The electronic circuit can govern the functioning of the pump 20, of the generator 15 powering the transducer(s) 13, or of the bubble generator 17, and can receive data from one or more sensors 35 such as a sensor for application of the handpiece to the skin. In the latter case, the control unit cannot start the pump 20, the emission of the acoustic waves and the bubble generation except for when the area to be treated is in place in front of the outlet 11 in a manner which will allow recycling of the composition.

Needless to say, the invention is not limited to the examples that have just been described.

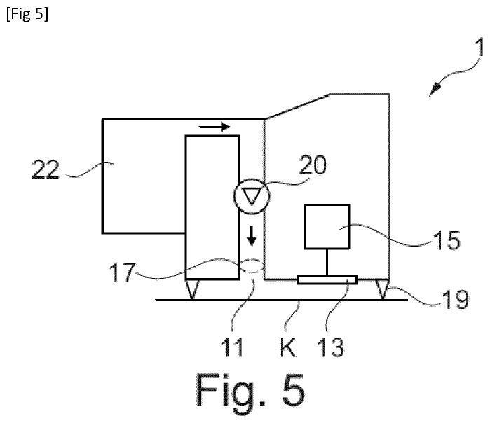

For example, the device may include, as illustrated in FIG. 5, an outlet 11 for dispensing the composition charged with bubbles and a transducer 13 arranged offset relative to the outlet 11. In this case, the composition applied to the area K to be treated, charged with bubbles, passes after moving the handpiece relative to the area to be treated, under the transducer 13 where it is exposed to the acoustic waves.

A spacing member 19, such as a flexible lip, may serve to distance the transducer 13 from the area to be treated, for example to prevent direct contact with the skin.

1-20. (canceled)

21. A process for cleansing keratin materials with a device configured to dispense a composition and emit acoustic waves, the method comprising:
(a) treating a region of keratin materials by applying a cosmetic composition comprising bubbles of a gas thereto with the device; and
(b) subjecting the treated region to acoustic waves with the device,
wherein the cosmetic composition is applied to the region continuously during the process.

22. The process according to claim 21, wherein the composition comprises at least one cleansing active agent, and wherein the total amount of cleansing active agent(s) is at least 0.02% by weight, relative to the total weight of the composition.

23. The process according to claim 21, wherein a frequency of the acoustic waves ranges from 0.3 kHz to 5 MHz, and a power of the acoustic waves ranges from 30 mW to 100 W/cm².

24. The process according to claim 21, further comprising:
(c) abrasing said region of keratin materials using abrasive particles simultaneously or sequentially with (a) and/or (b).

25. The process according to claim 21, wherein the cosmetic composition is at least partially recovered and recycled during the process.

26. The process according to claim 21, wherein step (a) occurs before step (b).

27. The process according to claim 21, wherein the treated region is an area of human skin comprising a makeup product.

28. The process according to claim 21, wherein the treated region is chosen from hair, nails, or the scalp.

29. The process according to claim 21, wherein the cosmetic composition comprises:
at least one foaming surfactant chosen from (i) polyoxyalkylenated alkyl(amido) ether carboxylic acid anionic surfactants, (ii) anionic surfactants different from the anionic surfactants of (i), (iii) nonionic surfactants, (iv) amphoteric/zwitterionic surfactants, or mixtures of two or more thereof; and/or
at least one compound chosen from (a) alkylpolysaccharides, (b) fatty alcohol ethers of polyethylene glycol, (c) oils, or mixtures of two or more thereof.

30. A device for cleansing keratin materials, comprising:
at least one applicator configured to dispense a composition via at least one outlet;
at least one chamber configured for the composition to circulate therein;
at least one ultrasonic transducer configured to emit acoustic waves;
at least one bubble generator configured to generate gas bubbles within the composition; and
at least one system configured to at least partially recover the composition.

31. The device according to claim 30, wherein the system comprises:
at least one pipe disposed proximate the at least one outlet, and
at least one pump configured to direct recovered composition through a filter and into the chamber.

32. The device according to claim 31, wherein the pipe is disposed around the outlet.

33. The device according to claim 31, further comprising a sealing member around the pipe, wherein the sealing member is configured to contain the composition and facilitate a return of the composition via the pipe.

34. The device according to claim 30, further comprising a reservoir configured to dispense the composition to the chamber.

35. The device according to claim 30, wherein a frequency of the acoustic waves ranges from 0.3 kHz to 5 MHz, and a power of the acoustic waves ranges from 30 mW to 100 W/cm².

36. The device according to claim 30, wherein the applicator comprises an abrasive surface.

37. The device according to claim 30, wherein the composition comprises:
at least one foaming surfactant chosen from (i) polyoxyalkylenated alkyl(amido) ether carboxylic acid anionic surfactants, (ii) anionic surfactants different from the anionic surfactants of (i), (iii) nonionic surfactants, (iv) amphoteric/zwitterionic surfactants, or mixtures of two or more thereof; and/or
at least one compound chosen from (a) alkylpolysaccharides, (b) fatty alcohol ethers of polyethylene glycol, (c) oils, or mixtures of two or more thereof.

38. The device according to claim 30, wherein the composition comprises at least one cleansing active agent, and wherein the total amount of cleansing active agent(s) is at least 0.02% by weight, relative to the total weight of the composition.

39. A process for cleansing skin with a device configured to dispense a composition and emit acoustic waves, the method comprising:
(a) treating a region of skin by applying a cosmetic composition comprising bubbles of a gas thereto with the device; and
(b) subsequently subjecting the treated region to acoustic waves with the device,
wherein the skin is skin to which a makeup product was previously applied.

40. The process according to claim 39, wherein the composition comprises at least one cleansing active agent, and wherein the total amount of cleansing active agent(s) is at least 0.02% by weight, relative to the total weight of the composition.
